(19) United States
(12) Patent Application Publication
(10) Pub. No.: US 2015/0313862 A1
(43) Pub. Date: Nov. 5, 2015

(54) "Pharmaceutical composition for reducing the trimethylamine N-oxide level"

(71) Applicant: Latvian Institute of Organic Synthesis, Riga (LV)

(72) Inventors: Ivars KALVINS, lkskile (LV); Maija DAMBROVA, Riga (LV); Edgars LIEPINS, Riga (LV); Elina MAKAROVA, Ogres district (LV); Osvalds PUGOVICS, Riga (LV); Solveiga GRINBERGA, Salaspils (LV); Eduards SEVOSTJANOVS, Jelgava (LV)

(73) Assignee: Latvian Institute of Organic Synthesis, Riga (LV)

(21) Appl. No.: 14/420,898

(22) PCT Filed: Oct. 24, 2013
(86) PCT No.: PCT/IB2013/059604

§ 371 Date: Feb. 10, 2015

(30) Foreign Application Priority Data
Oct. 25, 2012 (LV) P-12-163

(51) Int. Cl. A61K 31/205

(52) U.S. Cl. CPC A61K 31/205

(57) ABSTRACT

The invention discloses the use of pharmaceutical compositions containing 3-(2,2,2-trimethylhydrazinium)propionate dihydrate and pharmaceutically acceptable salts thereof for trimethylamine N-oxide level decrease in a body.

### FIELD OF THE INVENTION

This invention relates to the medicine, and namely to the use of a pharmaceutical composition containing Meldonium (3-(2,2,2-trimethyhydrazinium)propionate dihydrate) or pharmaceutically acceptable salts thereof.

### BACKGROUND OF THE INVENTION

One of the unresolved medical problems is the treatment of patients with renal failure, in particular at the terminal stages of the disease, when patients require a constant hemodialysis. It is well known that such patients often suffer from cardiovascular diseases (CVD) in addition to the main disease.

It is known that trimethylamine N-oxide (TMANO) accumulation is possible in the body of such patients (M. A. Bain, R. Faull, G. Fornasini, R. W. Milne, A. M. Evans. Accumulation of trimethylamine and trimethylamin-N-oxide in end-stage renal disease patients undergoing haemodialysis. Nephrol Dial Transplant (2006) 21: 1300-1304). However, only in 2011 it was found that a positive correlation exists between the patient's trimethylamin-N-oxide blood level with simultaneous suffering from one, two or three CVDs (Z. Wang, E. Klipfell, B. J. Bennet, R. Koeth, B. S. Levison, B. DuGar, A. E. Feldstein, E. A. Britt, Xiaoming Fu, Yoon-Mi Chung, Yuping Wu, P. Schauer, J. D. Smith, H. Alleayee, W. H. Wilson Tang, J. A. DiDonato, A. J. Lusis, S. L. Hazen. Gut flora metabolism of phosphatidylcholine promotes cardiovascular disease. Nature, 472, 57-63, (2011)). Therefore it turned into an actual problem to discover a medicament able to decrease the TMANO level in human blood, thus preventing the development of CDV not directly related to the lipid exchange.

Although it is known that TMANO blood level in animals may be decreased by the use of broad spectrum antibiotics, such treatment is not widely used for patients with chronic CVDs because of the antibiotics resistance problem and possible adverse side effects, and also because it was not proved that this approach may decrease the CDV recurrence (C. M. O'Connor et al., Azithromycin for the secondary prevention of coronary heart disease events—the WIZARD study: a randomized controlled trial. J. Am. Med. Assoc., 290, 1459-1466 (2003); C. P. Cannon et al., Antibiotic treatment of Chlamydia pneumonia after acute coronary syndrome. N. Engl. J. Med. 352, 1646-1654 (2005); R. Andraws, J. S. Berger, D. L. Brown. Effects of antibiotic therapy on outcomes of patients with coronary artery disease: a meta-analysis of randomized controlled trials. J. Am. Med. Assoc. 293, 2641-2647 (2005)).

An alternative approach includes an introduction of probiotic microorganisms (F. P. Martin et al., Probiotic modulation symbiotic gut microbial-host metabolic interactions in humanized microbiome model. Mol Syst. Biol., 4, 157 (2008)), however this approach was not clinically approved yet, and this is very important since human intestinal microflora by its content is substantially different from the intestinal microflora of rodents.

Thus, until now no drug has been known which is able to decrease the trimethylamine N-oxide level in human and which is useful as preventive medicament for health maintenance of those people who consume food of animal origin in large quantities.

Therefore, it is an actual problem to discover such pharmaceutical composition, which can provide a substantial and stable TMANO body level decrease, thus protecting from the increased risk of CVD, as well as eliminate the negative health effect of increased TMANO level resulted from other diseases, e.g. for the patients with renal failure.

### DESCRIPTION OF THE INVENTION

We have unexpectedly discovered that the cardiovascular medicament Meldonium, known already for its effect on energy metabolism, can substantially decrease the trimethylamine N-oxide level in human body, being administered in relatively low dosage.

Meldonium is a cardiovascular medicament widely used in the former USSR countries, which mechanism of action in the CVD therapy is based on the gamma-butyrobetaine hydroxylase inhibition and associated carnitine levels change both in blood and tissues (Dambrova M., Liepinsh E., Kalvinsh I., Mildronate. Cardioprotective Action through Carnitine-Lowering Effect, Trends Cardiovasc. Med., 2002: 275-279). Carnitine level decrease is connected with the decrease of fat acids β-oxidation rate and oxygen economy in ischemic tissues, what positively effects the treatment of ischemic-related diseases like cardiac failure and angina. Studies are also known where Meldonium effect on the sugar metabolism was described (Liepinsh E, Vilskersts R, Zvejniece L, Svalbe B, Skapare E, Kuka J, Cirule H, Grinberga S, Kalvinsh I, Dambrova M. Protective effects of mildronate in an experimental model of type 2 diabetes in Goto-Kakizaki rats. Br. J. Pharmacol. 2009, 157(8), 1549-1556). Similarly, there are indications that Meldonium could have an effect on the synthesis of nitric oxide in various tissues (Sjakste N, Gutcaits A, Kalvinsh I. Mildronate: an antiischemic drug for neurological indications. CNS Drug Rev. 2005; 11(2):151-68). Literature data exists regarding the Meldonium reduction of atherosclerotic plaque formation in pro-atherosclerotic animals that is associated with Meldonium effect on fatty acid metabolism (Vilskersts R, Liepinsh E, Mateuszuk L, Grinberga S, Kalvinsh I, Chlopicki, Dambrova M. Mildronate, a regulator of energy metabolism, reduces atherosclerosis in apoE/LDLR−/− mice. Pharmacology, 2009, 83(5), P. 287-293).

However, nothing was known about whether the use of Meldonium causes such changes in the levels of metabolites in the human body that are not related to the energy metabolism or NO regulated processes in the human body.

We have unexpectedly discovered that after 7 consecutive days of oral administration of a pharmaceutical composition containing Meldonium (2 times a day in a dose of 500 mg) to humans who eat the TMANO-rich food (W. J. Dyer, Amines in Fish Muscle. VI. Trimethylamine Oxide Content of Fish and Marine Invertebrates, Journal of the Fisheries Research Board of Canada, 1952, 8c(5):314-324, 10.1139/f50-020) results in statistically significant TMANO level reduction by 47% compared with TMANO level before the Meldonium use. At the same time we have found the statistically significant and substantial (about 25%) increase of urine excreted TMANO amount. This means that the Meldonium provides TMANO increased excretion or self-cleaning of this adverse metabolic product, which concentration in a body correlates with simultaneous suffering from several CVDs (Z. Wang, E. Klipfell, B. J. Bennet, R. Koeth, B. S. Levison, B. DuGar, A. E. Feldstein, E. A. Britt, Xiaoming Fu, Yoon-Mi Chung, Yuping Wu, P. Schauer, J. D. Smith, H. Alleayee, W. H. Wilson Tang, J. A. DiDonato, A. J. Lusis, S. L. Hazen. Gut flora metabolism of phosphatidylcholine promotes cardiovascular disease. Nature, 472, 57-63, (2011)).

With respect to the extremely low toxicity of Meldonium and low level of adverse side effects in patients, our discovery opens the way for Meldonium use to reduce TMANO levels in healthy subjects as well as in those patients whose disease pathogenesis mechanism may be related to increased blood levels of this metabolite, such as patients with renal failure who were subjected to hemodialysis during treatment of the main disease. Meldonium may be particularly useful as preventive medicament for patients with multiple CVDs. Meldonium can be used as a prophylactic TMANO level increase preventer for subjects consuming phosphatidylcholine-, betaine- and trimethylamine-rich products, as well as other nutrients which metabolism leads to trimethylamine N-oxide formation (B. A. Seibel, P. J. Walsh. Trimethylamine oxide accumulation in marine animals: relationship to acylglycerol storage. J Exp Biol. 205, 297-306, (2002)). This particularly applies to those categories of people who eat large amounts of animal products, such as athletes.

This invention is illustrated with the following examples, which should not be considered as limiting the application in any way.

Eight healthy volunteers were involved in the study, 4 women and 4 men. The study was conducted in accordance with the Declaration of Helsinki, and with bioethical norms approved by the Central Committee of Medical Ethics. All study participants have signed informed consent forms. Exclusion criteria for participation in the study were: the use of dietary supplements, smoking, pregnancy and alcohol addiction. At the study beginning information about the participant's age, medication use, height and weight was recorded.

Before the study, participants did not eat fish and fish products for 7 days. Then, a venous blood sample and evening and morning urine samples were collected from the study participants. At the first stage of experiment, all participants received 150 grams of fish for lunch, once a day, during 7 days, which served as a source of TMANO increased formation in the body (W. J. Dyer. Amines in Fish Muscle. VI. Trimethylamine Oxide Content of Fish and Marine Invertebrates, Journal of the Fisheries Research Board of Canada, 8c(5): 314-324, 10.1139/f50-020, 1952).

During the next stage of experiment, to determine the effect of Meldonium on TMANO concentration in blood plasma and urine, in addition to trimethylamine-rich food source, a pharmaceutical composition containing 500 mg of Meldonium was administered to each study participant during 7 days, twice a day (morning and evening). Blood samples were taken after 7 days of the trimethylamine-rich food diet and after 7 days of trimethylamine-rich food diet, accompanied by the Meldonium containing pharmaceutical composition administration. Urine samples were collected daily in the morning and in the evening during all 14 study days. Urine and plasma samples were stored at −20° C. until examination.

TMANO concentration in plasma and urine samples was determined by ultrahigh performance liquid chromatography-tandem mass spectrometry method. Chromatographic separation of samples was performed using Acquity chromatographic system (Waters), using Acquity HILIC BEH (2.1×100 mm, 1.7 μm) column (Waters). Liquid phase: acetonitrile—10 mM ammonium acetate (pH4) with linear gradient from 75% to 55% of acetonitrile, flow rate 0.25 ml/min, injection volume 5 μl. TMANO detection and quantification was performed using Quattro Micro™ spectrometer (Micromass) in an ion reaction monitoring (TMANO ion transition m/z75.8>m/z58.3, IS ion transition m/z175.4>m/z86.0) positive electrospraying mode (cone voltage 26V, collision energy 14 eV, collision gas—argon). Data collection and processing was performed with the MassLynx V4.1 software (Waters).

Participant's blood plasma samples (25 μl) were added to 500 μl of internal standard (IS) 3-(2,2-dimethyl-2-prop-1-yl-hidrazinium)propionate (200 ng/ml) solution in acetonitrile/methanol mixture (3:1), and after sample centrifugation (10 min, 13000 rpm) the supernatant was separated from sediment and injected into chromatography system.

Urine samples prior to analysis were diluted with deionized water in 1:50 ratio. The diluted samples (100 μl) were added to 700 μl of internal standard (IS) 3-(2,2-dimethyl-2-prop-1-yl-hidrazinium)propionate (200 ng/ml) solution in acetonitrile/methanol mixture (3:1), and after sample centrifugation (10 min, 13000 rpm) the supernatant was separated from sediment and injected into chromatography system.

TMANO concentration in the sample was calculated using the linear relationship TMANO peak area/IS peak area—TMANO concentration using the data processing software QuanLynx V4.1 (Waters).

### Creatinine Detection in Urine Samples

Creatinine concentration was calculated by slightly modified Jaffe method (Jaffe M. Ueber die Niederschlag, welchen Pikrinsaure in normalem Ham erzeugt and uber eine neue Reaction des Kreatinins. Z physiol Chem 1886; 10: 391-400). 135 μl of reaction mixture consisting of 1 part of 0.6% aqueous solution of picric acid and 5 parts of 1M NaOH (mixed immediately prior to measurement) was added to 15 μl of dilute urine samples or creatinine standard solutions (0-0.2 mg/ml). After 10 min of incubation at room temperature the absorbance was measured at 492 nm with a μQuant™ multiwell plate microscope spectrophotometer (Biotek Instruments, USA).

Data is presented as the mean±standard error mean (SEM). Student's t-test for testing the reliability results was used for the groups comparison. The total amount of urinary excretion of TMANO during 7 days was found calculating TMANO urinary concentrations of total area under the curve. The results are considered reliable if the p value was less than 0.05. Data collection and statistical analysis were performed used Microsoft Excel 2003, GraphPad Prism 3.0 (GraphPad Software Inc, USA) and SPSS 19.0 (SPSS, Chicago, Ill., USA) software.

### DESCRIPTION OF THE FIGURES

FIG. 1. TMANO concentration in the healthy volunteers' blood plasma. TMANO concentration was measured before the study, after TMANO source rich diet (7 days from the study beginning) and after TMANO source rich diet with simultaneous Meldonium (1 g/day) administration (14 days from the study beginning).

FIG. 2. Total amount of TMANO excreted with urine during 7 days. TMANO concentration in urine was measured two times a day before the study, after TMANO source rich diet (7 days from the study beginning) and after TMANO source rich diet with simultaneous Meldonium (1 g/day) administration (14 days from the study beginning).

### TMANO CONCENTRATION DETECTION

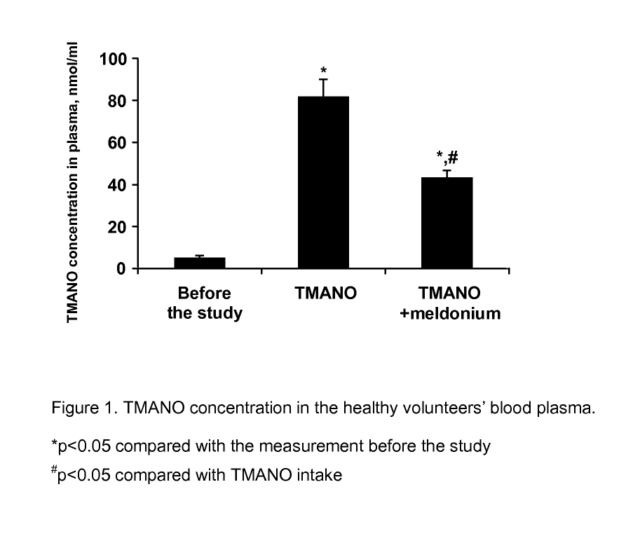

From the analyses results it was found that TMANO concentration in blood plasma of study participants before the experiment or at baseline was 4.9±1.3 nmol/ml. After 7 days of trimethylamine sources rich diet, TMANO concentrations statistically significantly increased to 81.5±8.6 nmol/ml. We have found that if TMANO source rich food was consumed together with Meldonium administration, TMANO plasma concentrations were statistically significantly reduced by 47% to 43.0±3.8 nmol/ml (FIG. 1, Table 1).

TABLE 1
TMANO concentration in study participant's blood plasma.

| | TMANO, |
|---|---|
| | nmol/ml |
| Before the study | 4.9 ± 1.3 |
| TMANO | 81.5 ± 8.6* |
| TMANO + Meldonium | 43.0 ± 3.8*^{,#} |

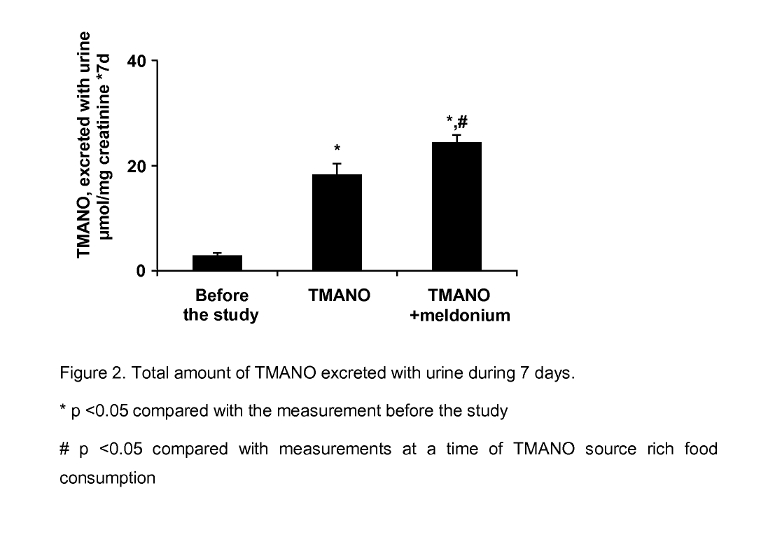

Using the TMANO concentration measurement in the urine of the study participants the total amount of excreted TMANO was determined. Before the experiment, study participants were excreting at average of 2.8±0.6 μmol TMANO to 1 mg of creatinine *7d. After the trimethylamine sources rich diet, average 7d with urine excreted TMANO amount was statistically significantly higher (18.2±2.2 μ/mol to 1 mg of creatinine *7d). For comparison, after the trimethylamine sources rich diet and Meldonium simultaneous administration, average 7d with urine excreted TMANO amount was statistically significantly even higher (24.3±1.5 to 1 mg of creatinine *7d) (FIG. 2., Table 2).

TABLE 2
Total excreted with urine TMANO amount during 7 days.

| | TMANO, μmol/mg |
|---|---|
| | creatinine *7 d |
| Before the study | 2.8 ± 0.6 |
| TMANO | 18.2 ± 2.2* |
| TMANO + Meldonium | 24.3 ± 1.5*^{,#} |

Thus, we have unexpectedly discovered that a pharmaceutical composition containing Meldonium reduces TMANO blood level and can be used for such diseases prevention and treatment, which pathogenesis is associated with increased levels of TMANO. Said diseases necessarily include atherosclerosis, in particular such arteriosclerosis forms, which are not accompanied with significant changes in cholesterol levels. It is also known that for patients in terminal stage of renal failure, who are treated with hemodialysis, Meldonium containing pharmaceutical compositions can be used to reduce TMANO level and the related risk of exposure to one or more cardiovascular diseases. Further, Meldonium-containing pharmaceutical compositions may also be used prophylactic to reduce the risk of developing one or more CVDs in people who consume food of animal origin rich in betaine, trimethylamine or other TMANO level enhancing components.

Meldonium-containing compositions can also be administered to people who consume other TMANO level enhancing compounds or have metabolic changes resulted in increase of the TMANO level, also to ensure TMANO increased excretion.

Thus, the pharmaceutical composition according to this invention allows providing a significant and sustained reduction of the TMANO concentration in the body, thus preventing the high risk of CVD development, as well as preventing the negative effects on human health of high TMANO level resulted from other diseases, such as in patients with renal failure.

Since Meldonium is easily soluble in water, to decrease TMANO levels an easy to prepare injection forms of Meldonium may be used, either in water or in physiological NaCl solutions, glucose or buffer solutions.

Because of its betaine-type structure, Meldonium is relatively easily absorbed also transdermally. Thus the pharmaceutical compositions according to this invention also can be prepared for transdermal or topical use in the form of patches, ointments, creams, gels, jelly, emulsions, solutions or other suitable pharmaceutical forms.

According to this invention, suitable pharmaceutical compositions of Meldonium for oral or sublingual administration may include but are not limited to coated or uncoated capsules, caplets, tablets, granules, pills, or a solution, syrup or other dosage forms for oral administration typically used in the art. These dosage forms can be prepared using conventional pharmaceutical compositions manufacturing methods known in the art. This invention also disclose pharmaceutical compositions containing as the active agent at least one of the non-hygroscopic Meldonium salts and a pharmaceutically acceptable solid and/or liquid excipient, typically used in the art for dosage forms manufacturing.

According to this invention, preferred compositions are those used for oral dosage forms preparation, as well as syrups and solutions, containing the compounds of this invention and/or their pharmaceutically acceptable salts and derivatives and pharmaceutically acceptable carriers.

For example, one of the illustrative pharmaceutical compositions of this invention, used for tablet production, include:

| | | |
|---|---|---|
| Meldonium | 500 | mg |
| Starch | 40 | mg |
| Talc | 20 | mg |
| Calcium stearate | 2 | mg |
| Total | 562 | mg |

One of the illustrative pharmaceutical compositions of this invention, used for capsules production, include:

| | | |
|---|---|---|
| Meldonium | 500 | mg |
| Lactose | 122 | mg |
| Starch | 52 | mg |
| Talc | 14 | mg |
| Calcium stearate | 6 | mg |
| Total | 694 | mg |

In case if Meldonium or a pharmaceutically acceptance salt thereof is administered as a composition for injection or drops, syrup or drink for oral administration, the pharmaceutical composition may contain Meldonium dihydrate or a pharmaceutically acceptable salt thereof according to this invention in total amount of 0.5% to 60% by weight and a pharmaceutically acceptable solvent, for example, but not limited to, distilled water, isotonic, glucose or buffer solution.

In case if the composition containing the active substance according to this invention is administered in tablets, caplets, pills, granules, powders or capsules, these may contain Meldonium or a pharmaceutically acceptable salt or derivative thereof in a total amount of 0.5 to 5 g per tablet, caplet, dragee, capsule or one dose of powder or granules.

Where the active substance is administered transcutaneously, it may be contented in an ointment or patch in amount of 0.5 to 40% by weight.

We claim:

1. A pharmaceutical composition for decrease of the trimethylamine N-oxide level in a human body, comprising 3-(2,2,2-trimethyhydrazinium)propionate (Meldonium) as an active ingredient, and a pharmaceutically acceptable carrier.

2. A pharmaceutical composition according to claim 1, comprising Meldonium in a form of 3-(2,2,2-trimethyhydrazinium)propionate dihydrate.

3. A pharmaceutical composition according to claim 1, comprising Meldonium in a form of a pharmaceutically acceptable salt.

4. A pharmaceutical composition according to any of the claims 1-3, characterized in that it is intended for oral or sublingual administration and it is in the form of covered or uncovered tablets, capsules, caplets, dragee, pellets, granules, powder, solution or syrup containing the active ingredient in amount of 0.1 to 5.0 g per single dose in case of a solid form, and in amount of 0.5-40% by weight in case of a liquid form.

5. A pharmaceutical composition according to any of the claims 1-3, characterized in that it is intended for parenteral administration and it is in the form of a sterile solution, containing the active ingredient in amount of 0.1 to 5.0 g per single dose along with a pharmaceutically acceptable solvent selected from the group comprising physiological NaCl solution, glucose solution and buffer solution of a pharmaceutically acceptable concentration.

6. A pharmaceutical composition according to any of the claims 1-3, characterized in that it is intended for transdermal administration and it is in the form of a patch, ointment, cream, gel, emulsion or solution containing the active ingredient in amount of 0.1 to 1.0 g per single dose.

7. A pharmaceutical composition according to any of the claims 1-3, characterized in that it contains 100 to 2500 mg of Meldonium.

8. A pharmaceutical composition according to any of the claims 1-3, characterized in that it is intended for parenteral administration and it is in a form of an injection solution containing Meldonium in amount of 50 to 400 mg/ml.

9. A pharmaceutical composition according to any of the claims 1-8 characterized in that it is used for prevention or treatment of such diseases which pathogenesis involves trimethylamine N-oxide level increase in a human body.

10. A pharmaceutical composition according to any of the claims 1-8 characterized in that it is used for prevention or treatment of diseases selected from the group comprising vascular endothelial dysfunction, atherosclerosis, and renal failure, when patient have an increased trimethylamine-N-oxide level in the body.
